(19) United States
(12) Patent Application Publication
(10) Pub. No.: US 2019/0205758 A1
(43) Pub. Date: Jul. 4, 2019

(54) GLAND SEGMENTATION WITH DEEPLY-SUPERVISED MULTI-LEVEL DECONVOLUTION NETWORKS

(71) Applicant: KONICA MINOLTA LABORATORY U.S.A., INC., San Mateo, CA (US)

(72) Inventors: Jingwen ZHU, Foster City, CA (US); Yongmian ZHANG, Union City, CA (US)

(73) Assignee: KONICA MINOLTA LABORATORY U.S.A., INC., San Mateo, CA (US)

(21) Appl. No.: 16/326,091

(22) PCT Filed: Dec. 13, 2017
(86) PCT No.: PCT/US17/66227

§ 371 Date: Feb. 15, 2019

Related U.S. Application Data
(60) Provisional application No. 62/441,156, filed on Dec. 30, 2016.

(51) Int. Cl. G06N 3/08; G06N 3/04; G16H 30/40; G06K 9/62; G06T 7/11; G06T 7/13

(52) U.S. Cl. CPC G06N 3/08; G06N 3/0454; G16H 30/40; G06K 9/628; G06K 9/6256; G06T 2207/20084; G06T 7/13; G06K 9/6277; G06K 9/6232; G06K 2209/05; G06T 2207/20081; G06T 7/11

(57) ABSTRACT

Pathological analysis needs instance-level labeling on a histologic image with high accurate boundaries required. To this end, embodiments of the present invention provide a deep model that employs the DeepLab basis and the multi-layer deconvolution network basis in a unified model. The model is a deeply supervised network that allows to represent multi-scale and multi-level features. It achieved segmentation on the benchmark dataset at a level of accuracy which is significantly beyond all top ranking methods in the 2015 MICCAI Gland Segmentation Challenge. Moreover, the overall performance of the model surpasses the state-of-the-art Deep Multi-channel Neural Networks published most recently, and the model is structurally much simpler, more computational efficient and weight-lighted to learn.

### BACKGROUND OF THE INVENTION

### Field of the Invention

This invention relates to artificial neural network technology, and in particular, it relates to deeply-supervised multi-level deconvolution networks useful for processing pathological images for gland segmentation.

### Description of Related Art

Artificial neural networks are used in various fields such as machine leaning, and can perform a wide range of tasks such as computer vision, speech recognition, etc. An artificial neural network is formed of interconnected layers of nodes (neurons), where each neuron has an activation function which converts the weighted input from other neurons connected with it into its output (activation). In a learning process, training data are fed into to the artificial neural network and the adaptive weights of the interconnections are updated through the leaning process. After learning, data can be inputted to the network to generate results (referred to as prediction).

A convolutional neural network (CNN) is a type of feed-forward artificial neural networks; it is useful particularly in image recognition. Inspired by the structure of the animal visual cortex, a characteristic of CNNs is that each neuron in a convolutional layer is only connected to a relatively small number of neurons of the previous layer. A CNN typically includes one or more convolutional layers, pooling layers, ReLU (Rectified Linear Unit) layers, fully connected layers, and loss layers. In a convolutional layer, the core building block of CNNs, each neuron computes a dot product of a 3D filter (also referred to as kernel) with a small region of neurons of the previous layer (referred to as the receptive field); in other words, the filter is convolved across the previous layer to generate an activation map. This contributes to the translational invariance of CNNs. In addition to a height and a width, each convolutional layer has a depth, corresponding to the number of filters in the layer, each filter producing an activation map (referred to as a slice of the convolutional layer). A pooling layer performs pooling, a form of down-sampling, by pooling a group of neurons of the previous layer into one neuron of the pooling layer. A widely used pooling method is max pooling, i.e. taking the maximum value of each input group of neurons as the pooled value; another pooling method is average pooling, i.e. taking the average of each input group of neurons as the pooled value. The general characteristics, architecture, configuration, training methods, etc. of CNNs are well described in the literature. Various specific CNNs models have been described as well.

Cancer grading is the process of determining the extent of malignancy in clinical practice to plan the treatment of individual patients. The advances in microphotograph and imaging enable acquisition of huge datasets of digital pathological images. The tissue grading invariably require identification of histologic primitives (e.g., nuclei, mitosis, tubules, epithelium, etc.). Manually annotating digitalized human tissue images is a laborious process, which is simply unfeasible. Thus, an automated image processing method for instance-level labeling of a digital pathological image is needed.

Glands are important histological structures that are present in most organ systems as the main mechanism for secreting proteins and carbohydrates. In breast, prostate and colorectal cancer, one of the key criterion for cancer grading is the morphology of glands. FIG. 4 shows a typical gland at different histologic grades from benign to malignant. A segmentation task is to delineate an accurate boundary for histologic primitives so that precise morphological features can be extracted for the subsequent pathological analysis. Unlike natural scene images which in general have well organized and similar object boundaries, the pathological images usually have large variances due to the tissues from different body parts and the aggressiveness level of the cancer so that they are more difficult to be learned from data approach to generalize to all unseen cases.

Recently, various approaches derived from Fully Convolutional Networks (FCNs) demonstrate remarkable results on several semantic segmentation benchmarks. See E. Shelhamer, J. Long, and T. Darrell, Fully Convolutional Networks for Semantic Segmentation, arXiv: 1605.06211v1, 2016. However, the use of large receptive fields and down-sampling operator in pooling layers reduces the spatial resolution inside the deep layers and blurs the object boundaries. FCN is well-suited for detecting the boundaries between two different classes; however, it encounters difficulties in detecting occlusion boundaries between objects from the same class, which are frequently present in pathological images. If FCN-based methods are directly applied to the pathological image segmentation tasks, the fine boundaries of tissue structure which are the crucial cues to obtain reliable morphological statistics are often blurred, as can be seen in FIG. 5. Most recently, DeepLab overcomes the drawbacks of FCNs and results in a new state-of-the-art at the PASCAL VOC-2012 semantic image segmentation task. See L-O Chen, G Papandreou, I Kokkinos, K. Murphy and A L. Yulile, DeepLab: Semantic Image Segmentation with Deep Convolutional Nets, Atrous Convolution, and Fully Connected CRFs, arXiv:1606.00915v2, 2017 (“L-O Chen et al. 2017”). However, Deeplab is not an end-to-end trained system, where the DCNN is trained first, and then a fully connected Conditional Random Field (CRF) is applied on top of the DCNN output as a constraint to compensate for a loss of localization accuracy due to downsampling in DCNNs.

Although significant progresses have been made in the last few years in using deep learning frameworks for image segmentation, there has been little effort to use deep frameworks for pathological image segmentation. This is mainly due to a lack of training data available in the public domain. Since the 2015 MICCAI gland segmentation challenge offered a benchmark dataset, several published works on gland segmentation with deep learning frameworks have been presented. Some work directly uses CNN trained as pixel classifiers, which is not ideal for image segmentation tasks compared with the image-to-image prediction techniques. A particularly interesting work is deep contour-aware network (DCAN) which is the winner of the 2015 MICCAI gland segmentation challenge. See H. Chen, X. Qi, L. Yu, and P. Heng, Dcan: Deep contouraware networks for accurate gland segmentation, IEEE Proceedings of the Conference of Computer Vision and Pattern Recognition (CVPR), pages 2487-2496, 2016 (“H. Chen et al. 2016”). DCAN uses a two independent upsampling branches to produce the boundary mask and object mask separately, and then fuses both results in the post-processing step. Arguably, the side output in DCAN up-samples directly from a low spatial resolution feature map by only using a single bilinear interpolation layer. Such an overly simple deconvolutional procedure is difficult to accurately reconstruct very fine and highly non-linear structure of tissue boundaries. More recently, Deep Multichannel Neural Networks uses a DCNN fusing the outputs from the three state-of-the-art deep models: FCN, Faster-RCNN and HED. The approach sets the state-of-the-art performance to a new level. However, the system is overly complex.

The recent success of DCNNs for object classification has led researchers to explore their feature learning capabilities for image segmentation tasks. In some of these models, the downsampling procedure which produces the low resolution representations of an image is derived from the VGG16 model with typically pre-trained weights by ImageNet dataset. The upsampling procedure that maps low resolution image representations to pixel-wise predictions varies among models. Typically, a linear interpolation procedure is used for upsampling low resolution feature map to the size of input. Such an over simple deconvolutional procedure can generally lead to loss of boundary information. To improve boundary delineation, there has been an increasing trend to progressively learn the upsampling layers from low resolution image representations to pixel-wise predictions. Several models require either MAP inference over a CRF or aids such as region proposals for inference. This is due to the lack of good upsampling techniques in their models.

In pathological analysis, before the arrival of deep networks, the segmentation methods mostly relied on hand engineered features including color, texture, morphological cues and Haar-like features for classifying pixels from histology images, and structured form models. These techniques often fail to achieve satisfactory performance in challenging cases where the glandular structures are seriously deformed. Recently, there have been attempts to apply deep neural networks for pathological image segmentation. They directly apply DCNNs for object classification to segmentation by classifying pixels of cell regions. Though their performance has already improved over methods that use hand engineered features, their ability to delineate boundaries is poor and extremely inefficient in terms of computational time during inference.

Consistent good quality gland segmentation for all the grades of cancer has remained a challenge. To promote solving the problem, MICCAI held gland segmentation challenge contest in 2015. Since then, the newer deep architectures particularly designed for pathological image segmentation have advanced the state-of-the-art in this field. For examine, in H. Chen et al. 2016, their model is derived from FCN by having two independent branches for inferring the masks of gland objects and contours. In the training process, the parameters of downsampling path are shared and updated for these two tasks jointly, while the parameters of upsampling layers for two branches are updated independently. The final segmentation result is generated by fusing both results in the post-processing step which is disconnected from the training of DCNN. Thus, the approach does not fully harness the strength of DCNN of learning rich feature representations. In addition, an observation can be made from their result that, the fuse of boundary information deteriorates the performance when applied on the challenging dataset of malignant cases.

More recently, Y. Xu, Y. Li, M. Liu, Y. Wang, Y. Fan, M. Lai, and E. Chang, Gland instance segmentation by deep multichannel neural networks, arXiv:1607.04889v2, 2016 describes a technique that uses three independent state-of-the-art models (channels): FCN as the foreground segmentation channel distinguishes glands from the background; Faster-RCNN as the object detection channel detects glands and their region in the image; HED model as the edge detection channel outputs the result of boundary detection. Finally, a DCNN fuses three independent feature maps output from the different channels to produce segmented instances. This approach pushed the state-of-the-art to a new level. Nevertheless, the system is overly complex.

S. Xie and Z. Tu, Holistically-nested edge detection, IEEE Proceedings of the International Conference on Computer Vision (ICCV), pages 1396-1403, 2015, describes an HED model where a skip-net architecture is employed to extract and combine multi-level feature representations. Thus, high-level semantic information is integrated with spatially rich information from low-level features to further refine the boundary location. Additional supervision is introduced to each side-output for better performance.

To summarize, unlike the semantic segmentation that a coarse segmentation may be acceptable in most of cases, pathological analysis needs instance-level labeling on a histologic image which generates highly accurate boundaries among instances. Existing deep learning methods in this field have limited capability to accurately reconstruct highly non-linear structure of tissue boundaries.

### SUMMARY

To mitigate limitations of existing technologies, embodiments of the present invention use a deep artificial neural network model that employs the DeepLab basis and the multi-layer deconvolution network basis in a unified model that allows the model to learn multi-scale and multi-level features in a deeply supervised manner. Compared with other variants, the model of the present embodiments achieves more accurate boundary location in reconstructing the fine structure of tissue boundaries. Test of the model show that it can achieve segmentation on the benchmark dataset at a level of accuracy which is significantly beyond the top ranking methods in the 2015 MICCAI Gland Segmentation Challenge. Moreover, the overall performance of this model surpasses the state-of-the-art Deep Multichannel Neural Networks published most recently, and this model is structurally much simpler, more computational efficient and weight-lighted to learn.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve these and/or other objects, as embodied and broadly described, the present invention provides an artificial neural network system implemented on a computer for classification of histologic images, which includes: a primary stream network adapted for receiving and processing an input image, the primary stream network being a down-sampling network that includes a plurality of convolutional layers and a plurality of pooling layers; a plurality of deeply supervised side networks, respectively connected to layers at different levels of the primary stream network to receive input, each side network being an up-sampling network that includes a plurality of deconvolutional layers; a final convolutional layer connected to output layers of the plurality of side networks which have been concatenated together; and a classifier connected to the final convolutional layer for calculating, of each pixel of the final convolutional layer, probabilities of the pixel belonging to each one of three classes.

In another aspect, the present invention provides a method implemented on a computer for constructing and training an artificial neural network system for classification of histologic images, which includes: constructing the artificial neural network, including: constructing a primary stream network adapted for receiving and processing an input image, the primary stream network being a down-sampling network that includes a plurality of convolutional layers and a plurality of pooling layers; constructing a plurality of deeply supervised side networks, respectively connected to layers at different levels of the primary stream network to receive input, each side network being an up-sampling network that includes a plurality of deconvolutional layers; constructing a final convolutional layer connected to output layers of the plurality of side networks which have been concatenated together; and constructing a first classifier connected to the final convolutional layer and a plurality of additional classifiers each connected to a last layer of one of the side networks, wherein each of the first and the additional classifiers calculates, of each pixel of the layer to which it is connected, probabilities of the pixel belonging to each one of three classes; and training the artificial neural network using histologic training images and associated label data to obtain weights of the artificial neural network, by minimizing a loss function which is a sum of a loss function of each of the side networks calculated using output of the additional classifiers and a loss function of the final convolutional layer calculated using output of the first classifier, wherein the label data for each training image labels each pixel of the training image as one of three classes including a class for gland region, a class for boundary, and a class for background tissue.

In a preferred embodiment, the primary stream network contains thirteen convolutional layers, five max pooling layers, and two Atrous spatial pyramid pooling layers (ASPP) each with 4 different scales, and each side network contains three successive deconvolutional layers.

In another aspect, the present invention provides a computer program product comprising a computer usable non-transitory medium (e.g. memory or storage device) having a computer readable program code embedded therein for controlling a data processing apparatus, the computer readable program code configured to cause the data processing apparatus to execute the above method.

It is to be understood that both the foregoing general description and the following detailed description are exemplary and explanatory and are intended to provide further explanation of the invention as claimed.

### BRIEF DESCRIPTION OF THE DRAWINGS

FIGS. 1A-E illustrate the architecture of a deep network according to an embodiment of the present invention. FIGS. 1A and 1B illustrate the network architecture for the prediction stage and training stage, respectively, and FIGS. 1C-E are enlarged views of three parts of FIG. 1B.

FIG. 2 schematically illustrates the training and prediction using the network.

FIG. 3 illustrates a qualitative comparison of performance of the model and method of FIGS. 1A-B with other models, in which the panels show: (a) ground truth; (b) segmentation result by FCN; (c) segmentation result by DeepLab basis; (d) predicted class score map by model and method of FIGS. 1A-B, where the green color indicates the boundary pixels; (e) segmentation result by the model and method of FIGS. 1A-B.

FIG. 4 illustrates examples of digital pathological images at different histologic grades. The top row shows images of cells at benign stage and malignant stage, respectively; the bottom row shows the respective ground truth of labeling for the images.

FIG. 5 illustrates the effect that fine boundaries of cell structure are often blurred when a FCN-based segmentation method is applied. The left panel is an original image; the middle panel is the ground truth image; and the right panel shows a segmentation result by using FCN.

### DETAILED DESCRIPTION OF PREFERRED EMBODIMENTS

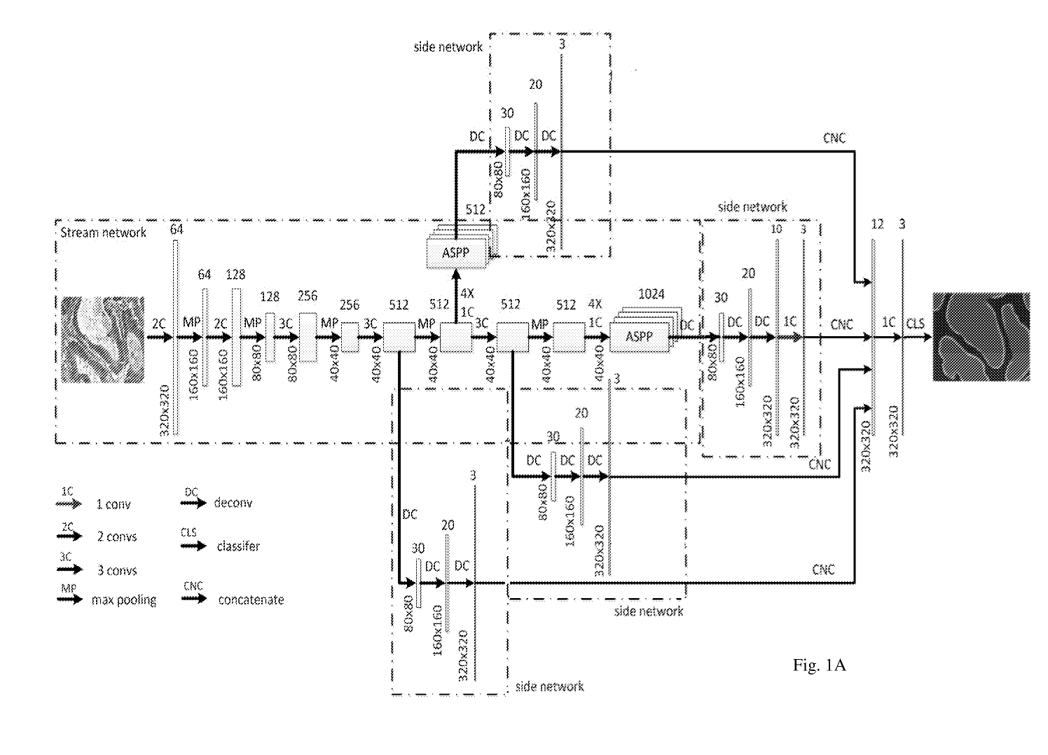

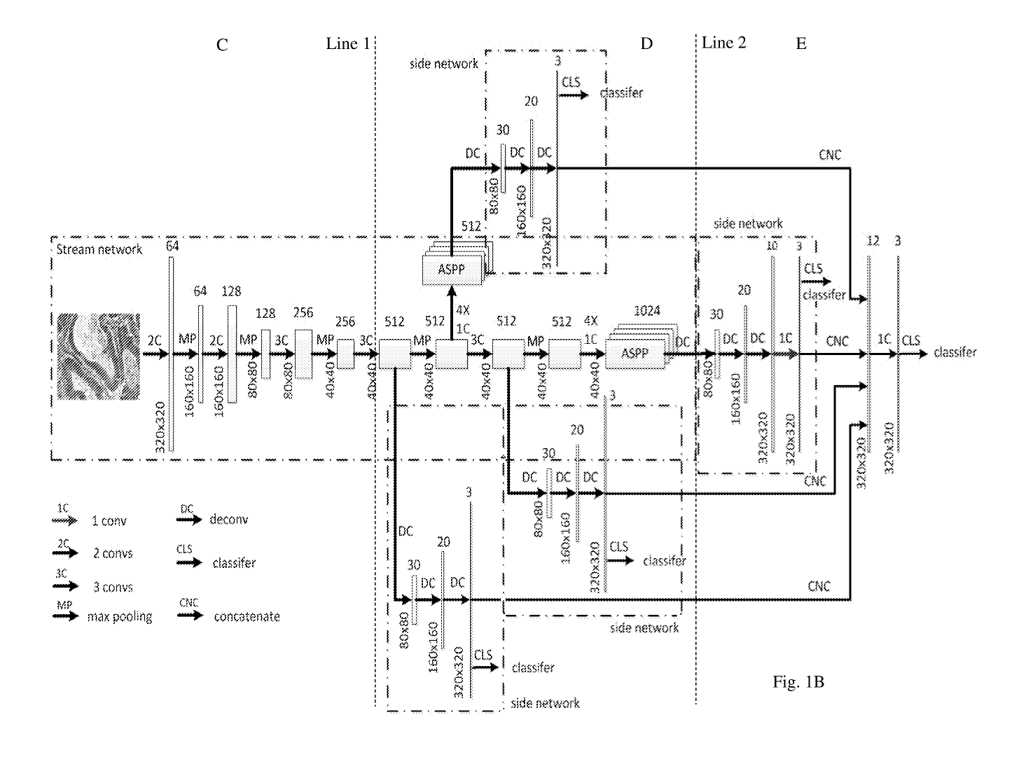

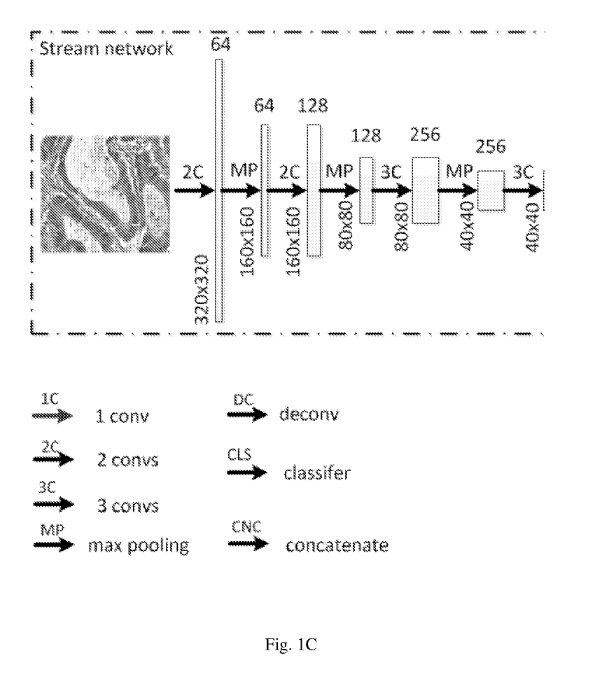

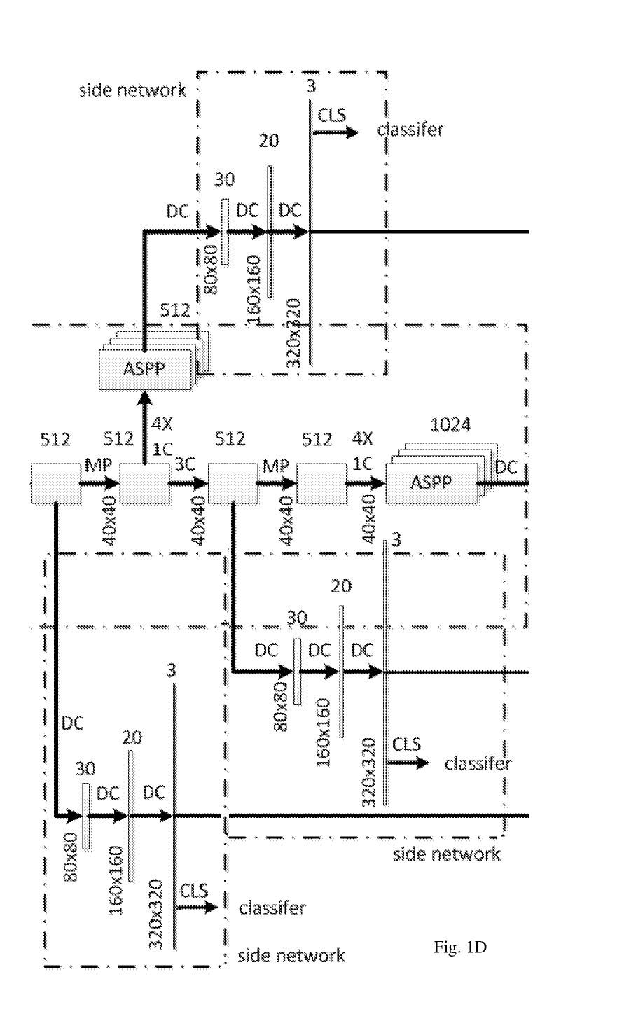

Similar to DCAN, the neural network model according to embodiments of the present invention is composed of a stream deep network and several side networks, as can be seen in FIGS. 1A-B. It, however, differs from DCAN in the following several aspects.

First, the model of the present embodiments uses DeepLab as a basis of the stream deep network, where Atrous spatial pyramid pooling with filters at multiple sampling rates allows the model to probe the original image with multiple filters that have complementary effective fields of view, thus capturing object as well as image context at multiple scales so that the detailed structures of an object can be retained.

Second, the side network of the model of the present embodiments is a multi-layer deconvolution network derived from the paper by H. Noh, S. Hong, and B. Han, Learning deconvolution network for semantic segmentation, published in arXiv:1505.04366, 2015. The different levels of side networks allow the model to progressively reconstruct highly non-linear structure of tissue boundaries. Unlike previous proposed technologies that use bilinear interpolation, the deconvolutional layers in the present model are trained in a deeply supervised manner to achieve accurate object boundary location.

Third, unlike DCAN that learns gland region and boundary in two separated branch upsampling module, the present model learns 3-class labels (gland region, boundary, background) simultaneously as a whole so that an error-prone procedure of fusing multiple outputs can be avoided.

The neural network model according to embodiments of the present invention also has similarity to the HED model described in S. Xie and Z. Tu, Holistically-nested edge detection, ICCV, 2015; a major difference between the model or the present embodiment and HED is in the way of upsampling, and network in HED is designed particularly for edge detection.

The present model achieved segmentation on the benchmark dataset of gland pathological images at a level of accuracy which is beyond previous methods.

A number of existing models to which the model of the present embodiments is related are described first.

DeepLab: Contrary to FCN which has a stride of 32 at the last convolutional layer, DeepLab produces denser feature maps by removing the downsampling operator in the last two max pooling layers and applying Atrous convolution in the subsequent convolutional layers to enlarge the receptive field of view. As a result, DeepLab has the following several benefits: (1) max pooling which consecutively reduces the feature resolution and spatial information is avoided; (2) the dense prediction map simplifies the upsampling scheme; (3) Atrous spatial pyramid pooling employed at the end of the network allows to explore multi-scale context information in parallel. A deeper network is beneficial to learn high-level features but comes at the cost of losing spatial information. Therefore, the Deeplab model with Atrous convolution is well-suited to meet the purpose of the model of the present embodiment.

Deconvolution Network: The deconvolution procedure for up-sampling is generally built on the top of CNN outputs. The FCN-based deconvolution procedure is fixed bilinear interpolation. Deconvolution using a single bilinear interpolation layer often causes the loss of the detailed structures of an object so that it is difficult to meet the requirement of the high accurate boundaries location. To mitigate the limitation, the approach of learning a deep deconvolution network is proposed in H. Noh, S. Hong, and B. Han, Learning deconvolution network for semantic segmentation, arXiv:1505.04366, 2015; and O. Ronneberger, P. Fischer, and T. Brox, U-net: Convolutional networks for biomedical image segmentation, arXiv:1505.04597v1, 2015. However, the original deep deconvolution network contains multiple series of unpooling, deconvolution and rectification layers, which is too heavy to train especially with very limited samples like the case of pathological image segmentation tasks. The model of the present embodiments modifies this feature.

The structure of the deep model for pathological image segmentation according to embodiments of the present invention is described in detail with reference to FIGS. 1A-E.

FIGS. 1A-E illustrate the architecture of a deep network according to an embodiment of the present invention. The model is composed of a primary stream network and several side networks. FIGS. 1A and 1B illustrate the network architecture for the prediction stage and training stage, respectively; they are identical except for the classifiers at the end of the side networks as will be explained in more detail later. FIGS. 1C-E are enlarged views of three parts of FIG. 1B; in FIG. 1B, the vertical lines labeled “Line 1” and “Line 2” are not parts of the model, but serve to indicate the division of FIG. 1B into three parts C, D and E that are enlarged in FIGS. 1C-E.

FIGS. 1A-E use symbols that are familiar to those skilled in the relevant art. For example, each rectangle box or vertical line represents a layer of the neural network, the number located above each layer represents layer depth, the numbers located near the bottom of each layer represent layer size, and the arrows represent the operations between layers. The meaning of each operation, such as convolution, max pooling, etc. are also familiar to those skilled in the relevant art and will not be described in detail here.

The model shown in FIGS. 1A-B is inspired by the HED model described in S. Xie and Z. Tu, Holistically-nested edge detection, ICCV, 2015. In order to learn rich hierarchical representations for accurate boundary location, the model of FIGS. 1A-B has one primary stream network and several deeply supervised side networks to perform image-to-image prediction. The stream network includes convolutional and max pooling layers to learn low-level and high-level contextual features, while each side network is composed of several deconvolutional layers for reconstructing the feature maps to object structure. Each side-output is associated with a classifier and concatenated together to feed into a convolutional layer at the end. The final convolutional layers learn to combine the outputs from different levels. The overall loss function includes the side networks loss and fusion loss at the end. The loss function is minimized via standard stochastic gradient decent as follows:

$${( W , {w}_{s} . {w}_{f} )}^{*} = arg min ( {\sum }_{m = 1}^{4} {l}_{s}^{( m )} ( W , {w}_{s}^{( m )} ) + {l}_{f} ( W , {w}_{s} . {w}_{f} ) )$$

where W, wₛ, w_{f} denote the weights for the stream network, the side networks, and the fusion layer (final convolutional layer), respectively. lₛ and l_{f} are the loss function for the side networks and the fusion layer at the end.

To utilize the strength of both DeepLab and Deconvolution network, the stream network of the model of FIGS. 1A-B is derived from the original DeepLab by replacing its bilinear interpolation with a learnable deep deconvolution networks for upsampling. Unlike the model described in H. Noh, S. Hong, and B. Han, Learning deconvolution network for semantic segmentation, arXiv:1505.04366, 2015, the deconvolution network in the model of FIGS. 1A-B discards the mirrored shape of CNN and un-pooling layers, and only contains a few consecutive deconvolutional layers and non-linear rectification layers, which is much shallower and weight-lighted to learn.

Down-sampling Module: The primary stream network (down-sampling module) contains 13 convolutional layers (2 groups of 2 consecutive convolutional layers and 3 groups of 3 consecutive convolutional layers), 5 max pooling layers, and two Atrous spatial pyramid pooling layers (ASPP) each with 4 different scales. ASPP is described in L-O Chen et al. 2017. Among the 5 max pooling layers, the first 3 max pooling layers reduce the spatial resolution of the resulting feature maps by a factor 2 consecutively, and the last 2 max pooling layers remove the downsampling operator to keep the resolution unchanged. This leads to the final convolutional layer which has a stride of 8 pixels. Compared with the original DeepLab model, the last two 1×1 convolutional layers and the following rectification layers and dropout layers at each sampling rate in ASPP are further removed from the original DeepLab model. The motivation behind is that, DeepLab is originally designed for natural image segmentation which contains thousands of classes, while the model of the present embodiment is designed for pathological images which have significantly fewer classes and thus do not require very rich feature representations.

Up-sampling Module: Each side network (up-sampling module) contains three successive deconvolutional layers. By setting the stride to 2 at each of the layers, the spatial resolution can be recovered to the original image resolution. The filter size is set as small as 4×4 to make it divisible by the stride to reduce checkerboard artifacts. There are several advantages of using a few small deconvolutional filters instead of a large one: (1) multiple small filters require fewer parameters; (2) a stack of small filters encode more nonlinearities; (3) consecutive deconvolution operations with small stride allow for recovery of fine-grained boundaries. This is particularly desirable for pathological image segmentation tasks. As the network goes deeper, it has more power to learn the semantic feature, but is less sensitive to the spatial variations so that it is difficult to generate pixel-level accurate segmentation. To address this issue, the side networks in the model of FIGS. 1A-B are connected to the different level of layers of the stream network so that the system progressively integrates high-level semantic information with spatially rich information from low-level layers during upsampling.

Class Labels: Although the multi-scale feature representation is sufficient to detect the semantic boundaries between different classes, it does not accurately pinpoint the occlusion boundaries due to the ambiguity in touching regions, and requires some post-processing to yield delineated segmentation. Due to the remarkable ability of CNN to learn low-level and high-level features, boundary information can be well encoded in the downsampling path and predicted in the end. Unlike DCAN that predicts boundary label and region label separately, the inventors believe that the feature channels of the downsampling module are redundant for learning ternary classes. To this end the model of FIGS. 1A-B uses a unified network that learns gland region, boundary and background simultaneously as a whole. The final score image labels each pixel to the three categories with resulting probability.

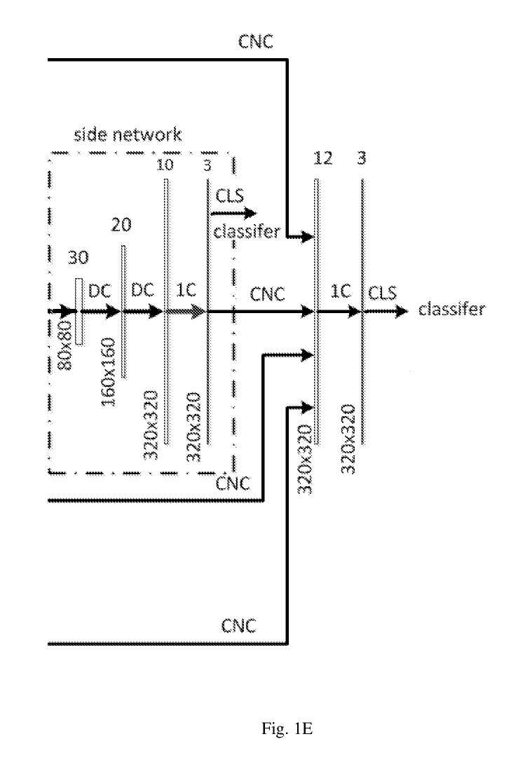

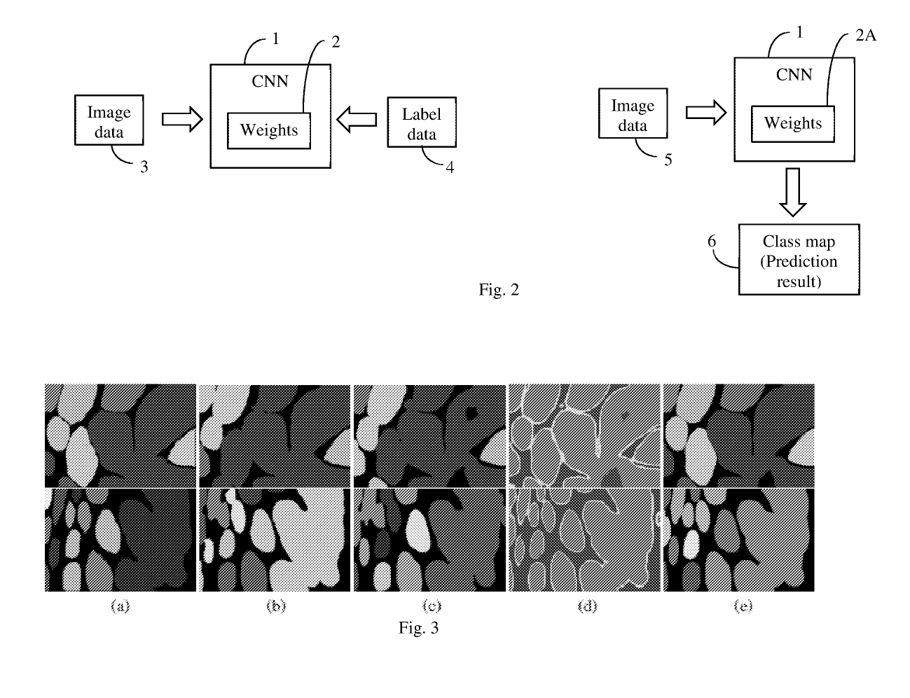

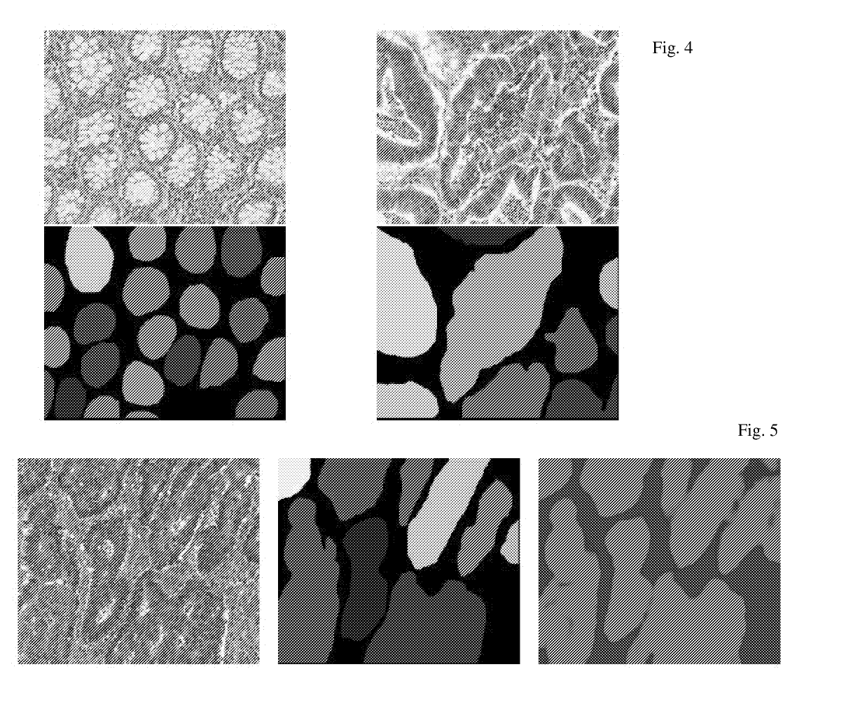

FIG. 2 schematically illustrates the process of training the network and using the trained network to process images (prediction). During the training stage, training data including training image data 3 and corresponding label data (ground truth) 4 are fed into the network 1 to learn the weights 2. During the prediction stage, image data to be processed 5 is fed into the network 2 containing the trained weights 2 to generate class maps (prediction result).

Note that FIG. 1B shows the network for the training stage and FIG. 1A shows the network for the prediction stage. They are identical except that the training stage model has a classifier output for each side network, which give four independent lose functions associated with the four individual classifiers. These loss functions are the lₛ components of the overall loss function that is minimized, as described in the above equation; the classifier associated with the final convolutional layer gives the l_{f} component of the overall loss function. For the prediction stage, the model has only one output, which is the probability map of 3 classes (shown at the far right end of FIG. 1A). I.e., the prediction stage model does not use the classifiers for the side networks.

The inventors have conducted a number of tests using the model shown in FIGS. 1A-B to process pathological images, described below.

MICCAI held gland segmentation challenge contest in 2015 and no competition has been held since. Presented below is the performance of the model of FIGS. 1A-B against the top 10 participants in the 2015 contest, and some of the state-of-the-art work published in 2016.

The dataset provided by MICCAI 2015 Gland Segmentation Challenge Contest was separated into Training Part, Test Part A, and Test Part B. The dataset consists of 165 labeled colorectal cancer histological images, where 85 images belong to training set and 80 images are used for testing. Test Part A contains 60 images including 33 in the histologic grade of benign and 27 in malignant. Test Part B contains 20 images including 4 in the histologic grade of benign and 16 in malignant. The details of dataset can be found in K. Sirinukunwattana, J. P. W. Pluim, H. Chen, X. Qi, P. Heng, Y. Guo, L. Wang, B. J. Matuszewski, E. Bruni, U. Sanchez, A. Bohm, O. Ronneberger, and B. Ben, Gland segmentation in colon histology images: The glas challenge contest, arXiv:1603.00275v2, 2016. Some examples of images of different histologic grades in the dataset are shown in FIG. 4. Considering the lack of large dataset, data augmentation is employed to enlarge the dataset and so to avoid overfitting. The augmentation transformations include pincushion and barrel distortion, affine transformation, rotation and scaling.

The network model of FIGS. 1A-B was implemented under Caffe deep learning library and initialized with a pre-trained from DeepLab. The model randomly cropped a 320×320 region from the original image as input and outputted the prediction class score map with three channels, i.e., gland region, boundary and background tissues. In the training phase, the learning rate was initialized as 0.001 and dropped by a factor of 10 at every step size of 10k iterations. The training stopped at 20k iterations.

Before the training procedure, boundary labels were generated by extracting edges from ground truth images, and the edges were dilated with a disk filter (radius 10). At the post processing step, the boundary and background channels were simply removed from the class score map to form a gland region mask. Then, an instance-level morphological dilation was employed on the region mask to compensate the pixel loss resulted from the removed boundaries to form the final segmentation result.

Using multi-perspective images is beneficial to the robustness in localizing boundaries. In the tests, two additional perspective images were used, which were generated by flipping the original image in top-down and left-right direction. The final predicted class score map is the normalized product of the class score maps resulted from the original image and two additional perspective images, respectively.

The inventors conducted tests to evaluate the efficacy of the present model of FIGS. 1A-B by comparing it with other architectures, including FCN and DeepLab basis. By “DeepLab basis”, it is meant that the procedure of Conditional Random Field (CRF) in the original Deeplab is not used. FIG. 3 illustrates a qualitative comparison of performance of the model and method of FIGS. 1A-B with some other models, in which the panels show: (a) ground truth; (b) segmentation result by FCN; (c) segmentation result by DeepLab basis; (d) predicted class score map by the present model and method, where the green color indicates the boundary pixels; (e) segmentation result by the present model and method. The examples shown in FIG. 3 demonstrate the segmentation quality of the present model against FCN and DeepLab basis. The result shows that the model of FIGS. 1A-B has clear boundaries in the touching regions in-between two gland regions which agree well with the ground truth, while both FCN and DeepLab basis approaches fail to predict the fine boundaries in-between objects of the same class due to using the linear interpolation procedure in upsampling.

The evaluation tool provided by the 2015 MICCAI Gland Segmentation Challenge was used to measure the model performance. The measuring methods provided by the evaluation tool include F1 score (which measures detection accuracy), Dice index (used for statistically comparing the agreement between two sets) and Hausdorff distance between the segmented object shape and its ground truth shape (which measures shape similarity). The measure was computed in an instance level by comparing a segmented instance against its corresponding instance of the ground truth.

Qualitative comparison using the above metrics, applied to test dataset Part A and Part B provided by MICCAI 2015 Gland Segmentation Challenge Contest, show that the present model outperforms FCN and DeepLab basis in all metrics. The performance of the present model is superior in part due to its learnable multi-layer deconvolution networks, while FCN and DeepLab basis use bilinear interpolation based upsampling without any learning.

The segmentation results using the present model and method were compared against the top 10 participants in the 2015 MICCAI gland segmentation challenge contest. Comparison shows that the present model outperformed all of the top 10 participants in all metrics with the only exception of the F1 score for dataset Part A the instant model underperformed one other model. The instant model surpassed the top 10 participants by a significant margin in terms of overall performance. Tests also show that the instant model outperforms in five of the six metrics compared to a more recent model known as deep Multichannel Neural Networks (DMNN) which obtained the state-of-the-art performance more recently. DMNN ensembles four most commonly used deep architectures, FCN, Faster-RCNN, HED model and DCNN, so the system is complex.

In summary, the model according to embodiments of the present invention is structurally much simpler, more computational efficient and weight-lighted to learn, while achieving high performance.

It will be apparent to those skilled in the art that various modification and variations can be made in the deeply-supervised multi-level deconvolution networks architecture and method of the present invention without departing from the spirit or scope of the invention. Thus, it is intended that the present invention cover modifications and variations that come within the scope of the appended claims and their equivalents.

What is claimed is:

1. An artificial neural network system implemented on a computer for classification of histologic images, comprising:
a primary stream network adapted for receiving and processing an input image, the primary stream network being a down-sampling network that includes a plurality of convolutional layers and a plurality of pooling layers;
a plurality of deeply supervised side networks, respectively connected to layers at different levels of the primary stream network to receive input, each side network being an up-sampling network that includes a plurality of deconvolutional layers;
a final convolutional layer connected to output layers of the plurality of side networks which have been concatenated together; and
a classifier connected to the final convolutional layer for calculating, of each pixel of the final convolutional layer, probabilities of the pixel belonging to each one of three classes.

2. The artificial neural network system of claim 1, wherein the primary stream network includes thirteen convolutional layers, five max pooling layers, and two Atrous spatial pyramid pooling layers (ASPP) each with four different scales, and each side network contains three successive deconvolutional layers.

3. The artificial neural network system of claim 2, wherein the primary stream network includes, connected in sequence: a first group of two consecutive convolutional layers, a first max pooling layer, a second group of two consecutive convolutional layers, a second max pooling layer, a third group of three consecutive convolutional layers, a third max pooling layer, a fourth group of three consecutive convolutional layers, a fourth max pooling layer, a fifth group of three consecutive convolutional layers, and a fifth max pooling layer,
the primary stream network further including a first ASPP with four different scales, connected after the fourth max pooling layer, and a second ASPP with four different scales, connected after the fifth max pooling layer,
wherein each of the first, second and third max pooling layers reduces a spatial resolution of its resulting feature maps by a factor of 2, and each of the fourth and fifth max pooling layers contains no downsampling operator and keeps a spatial resolution of its resulting feature maps unchanged.

4. The artificial neural network system of claim 3, wherein the plurality of side networks includes a first side network connected to a last one of the second group of three consecutive convolutional layers, a second side network connected to the first ASPP, a third side network connected to a last one of the fifth group of three consecutive convolutional layers, and a fourth side network connected to the second ASPP.

5. The artificial neural network system of claim 2, wherein each of the plurality of side networks includes three successive deconvolutional layers, each layer having a stride of 2, and wherein an output feature map of each of the plurality of side networks has a same spatial resolution as a spatial resolution of the input image.

6. The artificial neural network system of claim 1, wherein an output feature map of each of the plurality of side networks has a same spatial resolution as a spatial resolution of the input image.

7. A method implemented on a computer for constructing and training an artificial neural network system for classification of histologic images, comprising:
constructing the artificial neural network, including:
constructing a primary stream network adapted for receiving and processing an input image, the primary stream network being a down-sampling network that includes a plurality of convolutional layers and a plurality of pooling layers;
constructing a plurality of deeply supervised side networks, respectively connected to layers at different levels of the primary stream network to receive input, each side network being an up-sampling network that includes a plurality of deconvolutional layers;
constructing a final convolutional layer connected to output layers of the plurality of side networks which have been concatenated together; and
constructing a first classifier connected to the final convolutional layer and a plurality of additional classifiers each connected to a last layer of one of the side networks, wherein each of the first and the additional classifiers calculates, of each pixel of the layer to which it is connected, probabilities of the pixel belonging to each one of three classes; and
training the artificial neural network using histologic training images and associated label data to obtain weights of the artificial neural network, by minimizing a loss function which is a sum of a loss function of each of the side networks calculated using output of the additional classifiers and a loss function of the final convolutional layer calculated using output of the first classifier, wherein the label data for each training image labels each pixel of the training image as one of three classes including a class for gland region, a class for boundary, and a class for background tissue.

8. The method of claim 7, wherein the primary stream network contains thirteen convolutional layers, five max pooling layers, and two Atrous spatial pyramid pooling layers (ASPP) each with 4 different scales, and each side network contains three successive deconvolutional layers.

9. The method of claim 8, wherein the primary stream network includes, connected in sequence: a first group of two consecutive convolutional layers, a first max pooling layer, a second group of two consecutive convolutional layers, a second max pooling layer, a third group of three consecutive convolutional layers, a third max pooling layer, a fourth group of three consecutive convolutional layers, a fourth max pooling layer, a fifth group of three consecutive convolutional layers, and a fifth max pooling layer,
the primary stream network further including a first ASPP with four different scales, connected after the fourth max pooling layer, and a second ASPP with four different scales, connected after the fifth max pooling layer,
wherein each of the first, second and third max pooling layers reduces a spatial resolution of its resulting feature maps by a factor of 2, and each of the fourth and fifth max pooling layers contains no downsampling operator and keeps a spatial resolution of its resulting feature maps unchanged.

10. The method of claim 9, wherein the plurality of side networks includes a first side network connected to a last one of the second three consecutive convolutional layers, a second side network connected to the first ASPP, a third side network connected to a last one of the third three consecutive convolutional layers, and a fourth side network connected to the second ASPP.

11. The method of claim 10, wherein the plurality of side networks includes a first side network connected to a last one of the second group of three consecutive convolutional layers, a second side network connected to the first ASPP, a third side network connected to a last one of the fifth group of three consecutive convolutional layers, and a fourth side network connected to the second ASPP.

12. The method of claim 7, wherein an output feature map of each of the plurality of side networks has a same spatial resolution as a spatial resolution of the input image.
